(19) United States
(12) Patent Application Publication
(10) Pub. No.: US 2021/0369916 A1
(43) Pub. Date: Dec. 2, 2021

(54) TERMINALLY STERILIZED DEMINERALIZED BONE MATERIAL (DBM) AND SYSTEM AND METHODS FOR PROTECTING DBM AGAINST TERMINAL IRRADIATION

(71) Applicants: Kai Lo, Holmdel, NJ (US); Lawrence Shim, Holmdel, NJ (US)

(72) Inventors: Kai Lo, Holmdel, NJ (US); Lawrence Shimp, Burlington, WI (US)

(21) Appl. No.: 16/762,328

(22) PCT Filed: Nov. 7, 2018
(86) PCT No.: PCT/US2018/059619

§ 371 Date: May 7, 2020

Related U.S. Application Data
(60) Provisional application No. 62/582,489, filed on Nov. 7, 2017.

(51) Int. Cl. A61L 27/36; A61L 27/48; A61L 2/08; F26B 5/00

(52) U.S. Cl. CPC A61L 27/3608; A61L 27/48; F26B 5/005; A61L 2/081; A61L 2/087; A61L 27/3687

(57) ABSTRACT

Terminally sterilized demineralized bone material (DBM) and systems and methods for protecting DBM against damage caused by terminal irradiation are described. A method includes providing the DBM, which includes at least a collagen matrix and natural, non-collagenous protein. The DBM is soaked in a mixture of glycerol and a solvent to remove and replace water in the collagen matrix of the DBM. The DBM is dried, after soaking, to form a protected DBM.

### CROSS REFERENCE TO RELATED APPLICATION

This application is a national stage entry of International Patent Application No. PCT/US2018/059619 which was filed on Nov. 7, 2018 and claims the benefit of U.S. Provisional Application No. 62/582,489, which was filed on Nov. 7, 2017, and is incorporated by reference as if fully set forth.

### BACKGROUND

Demineralized bone matrix (DBM) may be prepared by removing the mineral portion of allograft bone tissue with acid, leaving a collagen scaffold and natural non-collagenous proteins. The collagen scaffold constitutes a natural abode for cells such that bone cells can grow on a surface thereof, making the collagen scaffold an ideal osteoconductive scaffold. Collagen fibrils may also be a nucleation site for mineral deposition. If processed properly, acid-stable, non-collagenous proteins consisting of bone morphogenetic proteins (BMPs) can retain their activity. These properties of the collagen scaffold and non-collagenous proteins make DBM osteoinductive, which means DBM is capable of inducing undifferentiated and pluripotent cells into bone forming cell lineage. In addition, DBM remodels after implantation in an osteotopic site, and, therefore, provides direct structural and functional connection between ordered living bone and the surface of the DBM graft, making it osteointegrative. Therefore, DBM is considered to be a very effective bone graft material if properly processed.

### SUMMARY

Terminally sterilized demineralized bone material (DBM) and systems and methods for protecting DBM against damage during terminal irradiation are described. A method includes providing the DBM, which includes at least a collagen matrix and natural, non-collagenous protein. The DBM is soaked in a mixture of glycerol and a solvent to remove and replace water in the collagen matrix of the DBM. The DBM is dried, after soaking, to form a protected DBM.

### BRIEF DESCRIPTION OF THE DRAWINGS

A more detailed understanding may be had from the following description, given by way of example in conjunction with the accompanying drawings, wherein like reference numerals in the figures indicate like elements, and wherein:

FIGS. 1A and 1B are diagrams of an unfolded polypeptide and a folded conformation in an aqueous environment;

FIG. 2 is a diagram showing a comparison between a dried or collapsed matrix and a wet expanded matrix;

FIG. 3 is a flow diagram of an example method of protecting a DBM against terminal irradiation;

FIG. 4 is a diagram of an example system for protecting DBM against terminal irradiation;

FIGS. 5A, 5B and 5C are graphs showing peak positions of solutions with 0% glycerol protectant, 20% glycerol protectant, and 50% glycerol protectant, respectively;

FIG. 6 is a graph showing protection of amide intensity with active protection technology when irradiated with gamma irradiation;

FIG. 7A is a graph showing the alkaline phosphate (ALP) assay of aseptic and terminally irradiated samples at 5 and 7 days;

FIG. 7B is a graph showing the mineralization assay of aseptic and terminally irradiated samples; and

FIGS. 8A, 8B and 8C are pictures showing a final DBM product that includes a 40%, 50% and 60% glycerol to bone concentration, respectively.

### DETAILED DESCRIPTION OF THE PREFERRED EMBODIMENTS

General processing steps to prepare DBM grafts may include thorough cleaning by chemical and/or mechanical means to eliminate cellular residue and pathogens, including viruses. Cleaned tissue may be subjected to demineralization and a series of possible chemical treatments and sterilization (if required or desired). While such processing may be necessary for proper preparation of DBM, the chemicals, mechanical processes and/or terminal sterilization processes may potentially damage both the collagenous and non-collagenous proteins, such as bone morphogenetic proteins (BMPs), in the DBM, diminishing their viability.

One method of DBM processing includes processing the allograft tissue aseptically, which may maintain viability of the DBM graft in a highly controlled environment without any terminal irradiation. While considered to be highly effective, aseptic processing is also extremely expensive as it requires specialized infrastructures and extensive sampling for sterility testing during production. Additionally, worldwide regulatory bodies are beginning to demand an expanded viral inactivation capability from the manufacturing processes of biologics.

Another method of DBM processing includes terminally irradiating grafts using terminal sterilization to ensure sterility of the product. Terminal irradiation may be leveraged to achieve a 10⁻⁶ sterility assurance level (SAL). In comparison to aseptic processing, the terminal irradiation processing makes the DBM processing much more tolerant to a higher bioburden load, which in turn demands much less infrastructural foot print to manufacture. This is because terminal sterilization only requires that bioburden be controlled to a relatively constant level and not be almost entirely eliminated as is required with aseptic processing.

While cheaper to implement, terminal irradiation may damage the collagen structure as well as the non-collagenous proteins of the DBM, making it ineffective compared to the aseptic grafts. In particular, sterilizing radiation is executed at an energy level where it becomes ionizing radiation. The impact of the radiation ejects electrons from the targeted molecules. The loss of electrons creates particles with missing electrons known as free radicals. These secondary irradiations may be electrically neutral particles that are highly reactive due to free, unpaired electrons. Small molecules, such as hydroxyl (.OH) and oxygen (.O) are the most commonly formed free radicals. These highly active free radicals may cause damage to chemical sites far removed from where they were formed. Accordingly, it may be desirable to control secondary free radical formation and reactivity in terminal irradiation of grafts for DBM processing in order to enable relatively inexpensive preparation of effective DBM samples.

The main source of hydroxyl radicals is water. Organic compounds containing hydroxide groups are lesser sources of hydroxyl radicals. The main source of oxygen radicals is free oxygen in air. Accordingly, radiation damage may be limited by minimizing the amount of water and oxygen present in the environment during radiation. Additionally or alternatively, radiation damage may be limited by lowering the temperature of the material during the radiation process. A lower temperature environment may limit the mobility of the free radicals and concomitant damages inflicted by the free radicals, though their rate of formation may be largely unchanged due to extremely high energy of the ionizing radiation particles. While these methods may limit radiation damage for sensitive materials, they may not be effective enough to provide optimal protection for a sensitive material, such as DBM.

Embodiments described herein provide for a system and methods for relatively inexpensively processing and terminally sterilizing sensitive proteins, such as DBM, while maintaining their efficacy, for example by penetrating an active protectant into the matrix of the graft by a suitable solvent. Such active protection of grafts may work in a number of different ways to protect the graft during processing, sterilization and storage.

While embodiments are described herein in terms of processing DBM, and, more specifically, bovine tissue DBM, one of ordinary skill in the art will recognize that the systems and methods described herein may be applicable to any tissue based allograft, including, for example, soft tissues, such as skin grafts, tendons, and fascia, and hard tissues, such as collagen based grafts, demineralized bone matrices, or combinations thereof. This may also be extended to ceramic based grafts, non-demineralized allografts or any xenograft to protect a sensitive protein or factor that can be incorporated on or with the graft matrix. It may also be applicable to protect releasable protein or cytokine based drugs, pharmaceuticals, proteins or peptide enhanced devices or matrices. The embodiments described herein may also be applicable, for example, for protection of DBM, sensitive biologics, proteins, factors, and drugs during processing and during terminal irradiation to maintain their efficacy.

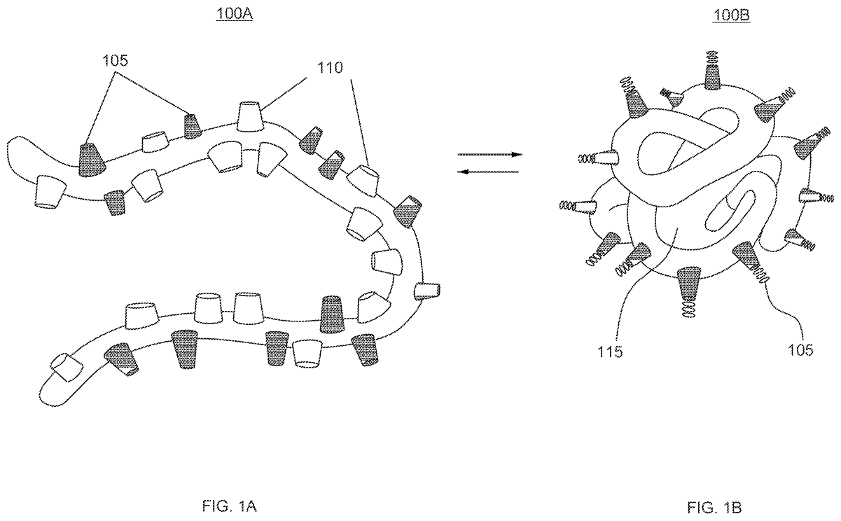

FIGS. 1A and 1B are diagrams of an unfolded polypeptide 100A and a folded conformation 100B in an aqueous environment. As shown in FIG. 1B, native proteins in a biological matrix or in solutions may be stabilized to their natural conformation through a hydrogen bonding mechanism with hydrophilic side chains 105 on the outside and the hydrophobic core 115 packed inside. In the example illustrated in FIG. 1B, the hydrophilic side chains 105 on the outside are polar side chains and the hydrophobic core 115 contains nonpolar side chains. The polar side chains 105 and the nonpolar side chains 110 can be seen more clearly in the unfolded polypeptide 100A of FIG. 1A.

The polar side chains 105 on the outside of the molecule of FIGS. 1A and 1B may form hydrogen bonds to water. Similarly, in properly processed, demineralized allograft tissue, water molecules may play a role in stabilizing the structural characteristics of the fibrous proteins, such as collagen, as well as the conformation of the globular proteins, such as non-collagenous bone morphogenetic proteins, through hydrogen bonding. This internal water may allow the proteins to retain their native physiological structure and functions, maintaining graft viability. However, the internal water may be detrimental to the viability of the native proteins during terminal sterilization or storage. Therefore, tissue processors and graft manufacturers may lyophilize or vacuum dry the grafts to eliminate the water in the matrix and reduce damage during sterilization or storage.

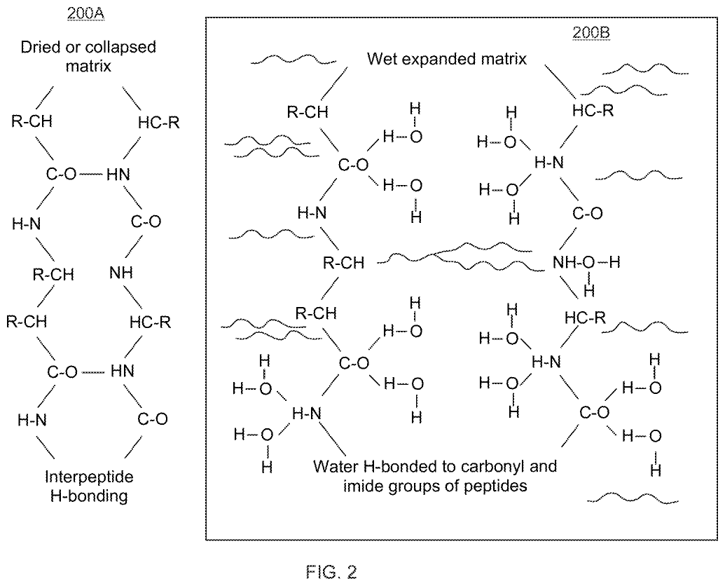

FIG. 2 is a diagram showing a comparison between a dried or collapsed matrix 200A and a wet expanded matrix 200B. As illustrated on the right side of FIG. 2, in the native state of a biological matrix, water molecules hydrate the polar functional groups of the N—H bonds of peptide molecules forming bound collagen water and maintaining the natural conformation of the protein molecules. As illustrated on the left side of FIG. 2, indiscriminate drying of the matrix collapses the collagen molecules as the adjacent collagen molecules form rapid, interpeptide hydrogen bonds among themselves. This kind of change in conformation may alter the natural healing properties of the scaffold and may affect the osteoinductivity and osteoconductivity of the scaffold. It may also be difficult to completely dry the matrix due to very strong hydrogen bonds between the water and peptide molecules.

As mentioned briefly above, active protection technology may be used to preserve the natural conformation and viability of the proteins in multiple ways in order to protect sensitive proteins during processing, sterilization and storage. Terminal irradiation and storage may be possible by introducing a protectant around the protein structure and simultaneously eliminating water from the system. Both globular proteins and fibrous protein structures may be stabilized by such a protectant. This may be done, for example, by exposing DBM to a mixture of a solvent and an active protectant, as described in more detail below. In embodiments, a concentration of the active protectant in the mixture may be chosen to optimize protection of the proteins post sterilization with terminal irradiation.

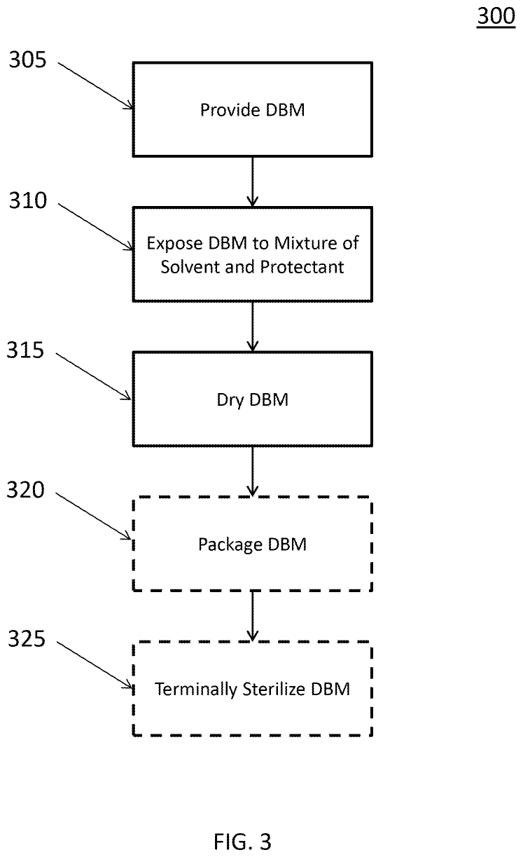

FIG. 3 is a flow diagram 300 of an example method of protecting a DBM against terminal irradiation. In the example illustrated in FIG. 3, the method includes providing a DBM (305). This may include preparing the DBM, using pre-prepared DBM, or any other method of providing DBM that may be understood by one of ordinary skill in the art. As mentioned above, the DBM may include at least a collagen scaffold or matrix and natural, non-collagenous proteins and may, for example, be formed by removing the mineral portion of allograft bone tissue.

The DBM may be exposed to a mixture of a solvent and an active protectant to remove and replace water from the collagen matrix of the DBM (310). In embodiments, the DBM may be covered, soaked or otherwise exposed to the mixture in such a manner that the solvent displaces water by penetrating deeply inside the structure, which may fully dry the tissue, and the active protectant is introduced within the protein structure. In embodiments, sensitive drugs, factors, proteins, cytokines, or other like materials, may be introduced into the protectant material if necessary or desired. Water and other volatiles, including the solvent, may also be removed if necessary or desired. The DBM may then be dried (315). The dried DBM may be packaged (320) if necessary or desired. The DBM or packaged DBM may be terminally sterilized (325), such as by irradiating or exposing the DBM or packaged DBM to gamma or electron-beam radiation. In embodiments, the irradiation may be performed in an inert environment, vacuum packed at a subzero temperature.

Choice of a suitable protectant and an effective non-aqueous solvent may allow the displacement of water molecules from around the protein. As a result, the biological factors may retain their native physiological structure and functions even though the entire environment is no longer an aqueous one. By eliminating water and its potentially damaging free radical reactions, the proteins may retain their natural functional properties better in this actively protected environment when they are terminally radiated. As this process removes water from the DBM and replaces it in order to maintain the structure of the DBM during terminal radiation, it should be noted that, for water to be removed from the DBM, the DBM should have bonded water in the matrix. Otherwise, the matrix may collapse and may not be able to be re-built by reintroduction of water or the protectant.

A suitable solvent should be miscible with water to penetrate deep within the structure as well as to dissolve the protectant. A suitable protectant may diffuse and penetrate deep within the protein structure with the help of the solvent and stabilize the protein after drying. The solvent may be a single solvent or a mixed solvent to impart a spectrum of properties. The choice of the solvent may be driven by its hygroscopicity, surface tension, solubility parameters, polarity, and miscibility with the protectant as well as its vapor pressure. Example solvents, and related properties, are provided in TABLE 1 below. Depending on the polarity and miscibility of the solvent, it may act as an excipient to incorporate different pharmaceuticals or other factors in the graft/matrix.

TABLE 1

| Substance | δ_{d} | δₚ | δₕ | δₜ |
|---|---|---|---|---|
| Acetone | 13.0 | 9.8 | 11.0 | 19.7 |
| Ethanol | 12.6 | 11.2 | 20.0 | 26.1 |
| Methanol | 11.6 | 13.0 | 24.0 | 29.7 |
| Water | 12.2 | 22.8 | 20.4 | 48.0 |
| Wet collagen | 11.8 | 15.3 | 22.5 | 30.1 |
| Dry collagen | 11.7 | 12.1 | 14.8 | 22.5 |

In embodiments, the chosen solvent may be prepared by varying component ratios to vary the solvent properties. For example, the component ratios may be chosen for the solvent such that the solvent may dissolve and incorporate a hemostat, which may form a blood clot and start the healing process. Examples of hemostats may include coagulation initiators, platelet activators, vaso-constrictors and fibrinolysis inhibitors. Other examples may include epinephrine, thrombin, fibrin, fibrinogen, and chitosan. Some polyols, such as polyethylene glycol (PEG), may have hemostatic properties of their own.

Additionally or alternatively, a mixed solvent may be prepared by varying the component ratios of the solvent, resulting in a solvent with a varied polarity and dissolution ability. Such a mixed solvent may be leveraged, for example, to dissolve a wide range of solutes, including, for example, different proteins or pharmaceuticals. As an example, different protectants, such as PEG, in different molecular weights may be introduced within the matrix by mixing with a suitable solvent such as alcohol. PEG may act as an excipient for different pharmaceuticals and, thus, may be introduced intimately within the matrix for controlled release. This may be helpful, for example, in surgery, by incorporating prophylactic antibiotics or by incorporating additional or non-native growth factors.

The free water and the collagen water mentioned above may be replaced by an anhydrous solvent to prevent the chemical degradation of matrix proteins and other proteins during irradiation and/or storage. Further, the physical conformation of the collagen molecules may be maintained by replacing the water molecules with suitable non-toxic, low reactivity protectant molecules. Further, to be able to replace water from the structure, the solvent should have a solubility parameter comparable to water.

Therefore, in embodiments, diffusion of an anhydrous protectant, such as glycerol, propylene glycol, or ethylene glycol, completely within the matrix of the demineralized bone tissue or any other tissue, such as skin or other processed tissue such as a collagen matrix, may be achieved by mixing the protectant with an anhydrous but water miscible solvent, such as ethanol or isopropanol. Other anhydrous solvents, such as methanol and acetone, may also be used. The choice of protectant may be varied depending on the final use as well as the relevant protein type that is required to be preserved during processing, sterilization and storage.

In embodiments, the protectant may be polyols, such as glycerol, propylene glycols, polyhydric compounds, such as propane 1,2 diol, propane 1,3 diol, ethylene glycol, erythritol, xylitol, sorbitol, mannitol, inositol, sugars, such as deoxyribose, ribose, arabinose, xylose, glucose, mannose, maltose, lactose, or sucrose, trehalose, mannitol, sucrose, or other suitable chemicals such as Dimethyl Sulfoxide (DMSO). In its native state, bound collagen water retains the structure between interpeptide bonds. Collagen water also creates a diffusion channel by keeping the interfibrillar space open. Further, molecules less than 6 kDa may easily diffuse between collagen fibrils to enter the collagen water.

Therefore, a protectant and a solvent for the active protection system should be chosen carefully based on the proteins being protected, polarity, size, toxicity, and other factors depending on end use.

As an example, ethanol or isopropanol may be chosen as a suitable polar solvent with a low surface tension and a high solubility parameter. For an active protection system, glycerol may be chosen as a protectant. In an example active protection system, ethanol or isopropanol, due to their low surface tension and high solubility parameters, may dissolve a polyol protectant, such as glycerol, to lower the surface tension of the same, allowing it to better diffuse and penetrate within the matrix. Also, since the molecular weight of glycerol is much less than 6 kDa (˜92.904 Da), it may easily penetrate the interstitial space of collagen water to stabilize the collagenous and non-collagenous proteins (such as BMP growth factors). Post lyophilization/drying, the solvent and remnants of water may be driven off to form an anhydrous polyol stabilized scaffold. The polyol infiltration process may be considered an active protection process that offers protection during and after processing and terminal irradiation and during storage. This stabilization may lead to more robust grafts that may retain their efficacy post terminal irradiation since the process may protect both globular and fibrous proteins.

In embodiments, the mixture may be liquid or may be a supercritical fluid. A supercritical fluid may be formed, for example, by cooling the mixture to a very low temperature while still maintaining a liquid state. Use of a supercritical fluid as the mixture may help to further preserve the proteins or other fragile and/or unstable additives, such as non-stable proteins or pharmaceuticals, during processing. In some embodiments, the graft may additionally or alternatively be packed under vacuum or inert gas and cooled during irradiation to minimize free radical damage.

If additional strength, or a longer resorption time, is required or desired for the tissue product, the tissue product may be cross-linked. Cross-linking may, for example, use sugars incorporated within the structure described above. Sugar cross-linking, which mimics the natural cross-linking process associated with aging, may be one example of a relatively benign cross-linking process. This may not significantly affect the active molecules bound to the collagen, which may help maintain the efficacy of the graft. In embodiments, a polyol may be mixed with polyacidic groups, such as sebaceic acid or citric acid, to polymerize within the matrix to form a cross-linked biodegradable matrix that may not be lavaged out. In other embodiments, an alcohol or other anhydrous solvent may be used to dissolve polyacidic groups, such as citric acid, and mixed with glycerol. A graft incorporating this solvent system may be vacuum dried and, post drying, may be heated up to approximately 90° C. to form a crosslinked DBM structure.

In embodiments, the soaking the DBM in the mixture of glycerol and the solvent may be performed under pressure. This may support better penetration of the mixture into the DBM. The soaking may be performed, for example, under a pressure of 40-100 kPa.

In embodiments, the packaging (320) may include packaging the DBM into a formed structure. In embodiments, a reinforcing biodegradable mesh may be disposed between the collagen or DBM tissue with enhanced bonding by added polyacidic groups and polyols. A formed structure, with or without the mesh, may be in the form of a strip, disc, wrap, sheet or any other suitable form for procedure specific applications. In embodiments, the structure may be custom formed according to the contour of patient-specific defect sites with the help of a computerized tomography (CT) scan augmented mold. In embodiments, the actively protected tissue material may be packaged by disposing it in a biodegradable mesh bag for better delivery.

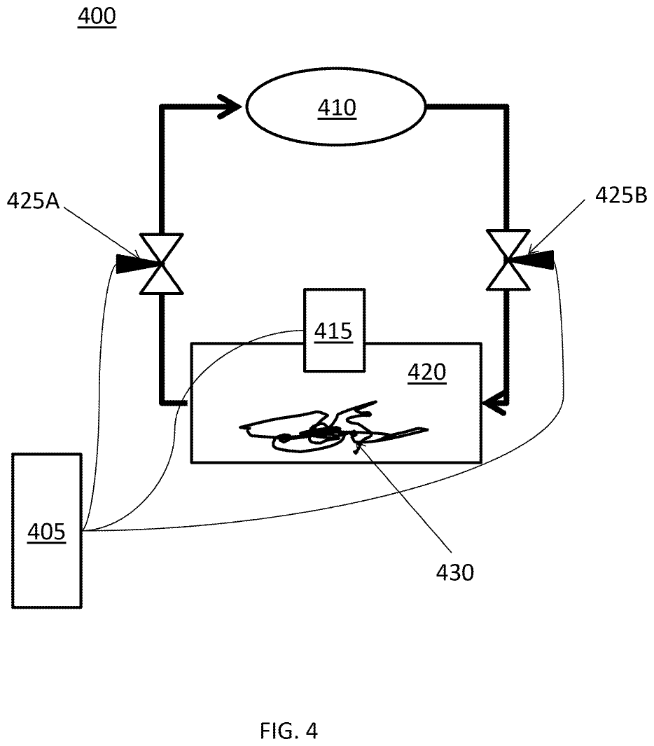

FIG. 4 is a diagram of an example system 400 for protecting DBM against terminal irradiation. In the example illustrated in FIG. 4, the system 400 includes a controller 405, a container 410, a drier 415, a reaction chamber 420 and valves 425A and 425B. The controller 405 may be any type of controller, such as a programmable logic controller (PLC). The drier 415 may be any suitable drier, such a lyophilizer. A mixture, such as any of the mixtures of a solvent and an active protectant described herein, may be introduced into the container 410, and one or more DBM fibers may be introduced into the reaction chamber 420. The controller 405 may be communicatively coupled to the valves 425A and 425B and may introduce at least some of the mixture from the container 410 into the reaction chamber 420 by controlling the valves 425A and 425B. In embodiments, the controller 405 may soak or otherwise cover the DBM 430 in the reaction chamber 420 by controlling the valves 425A and 425B. The reaction that takes place in the reaction chamber 420 may remove and replace the water in the collagen matrix of the DBM 430. After the water is removed and replaced, the controller 405 may remove any remaining mixture from the reaction chamber by controlling the valves 425A and 425B. The controller 405 may control the drier 415 to dry the DBM 430, resulting in protected DBM.

Different active protection systems with a wide range of properties may be designed depending on end use. Experiments are described below in which a system of alcohol (ethanol or isopropanol) and protectant (glycerol) is chosen to demonstrate the protective properties of the active protection system on bovine DBM, which is a collagenous structure with a mix of native, non-collagenous proteins, such as BMP, transforming growth factor (TGF)-β, and vascular endothelial growth factor (VEGF). Experiments were conducted to demonstrate the effective range of protectant concentration in the active protection system. Further, the effectiveness of the system is demonstrated by testing the osteoinductive properties of the DBM post sterilization using aseptically processed DBM as control. Fourier transform infrared spectroscopy (FTIR) analysis is used to demonstrate the viability of the collagen protein before and after sterilization.

FTIR analysis on an organic sample may allow for identification of the characteristic peak or peaks for the particular moiety of interest. Collagenous and non-denatured non-collagenous proteins in the DBM, such as BMP, and TGF-β, are considered viable in their native conformation or intact secondary structure. In this example, collagen was focused on as it is much more abundant compared to non-collagenous proteins in bone tissue, which facilitates the quantification of the relevant peaks. Successful protection of collagen molecules may also indicate that the non-collagenous proteins are also protected. Further in vitro tests also corroborate the protection of the activity of the non-collagenous proteins.

Among many vibrational bands in FTIR of collagen, the amide I band at wave number ˜1656-1657 cm⁻¹ (peptide bond >C═O stretch) is especially sensitive to the secondary structure of collagen. During denaturation of collagen, the intensity of this peak is lowered. Therefore, a ratio of the peak intensities of the peak of the same sample before and after gamma irradiation provides a measure of denaturation of the proteins in the graft as a result of the gamma irradiation (25-35 kGy).

In this experiment, samples of DBM from the same batch of tissue with active protection were prepared by using glycerol as a protectant. A series of solutions from 10% to 90% (v/v) of glycerol concentration were prepared using isopropyl alcohol as a solvent. Two additional sample groups without the protection technology (0% and 100% glycerol concentration) were also prepared to compare with the protected samples. All samples were vacuum dried at low temperature, packaged and gamma sterilized in presence of dry ice at a dose of 25-35 kGy.

Post irradiation, FTIR analysis of all the samples was carried out by an FTIR instrument (IR 200 by Thermo Electron North America), using about 0.1-0.3 gms samples from each group. The intensity for the area under the curves was found out from integrating the relevant peak. Percentage drops (or gains) in intensity as a result of gamma irradiation were calculated.

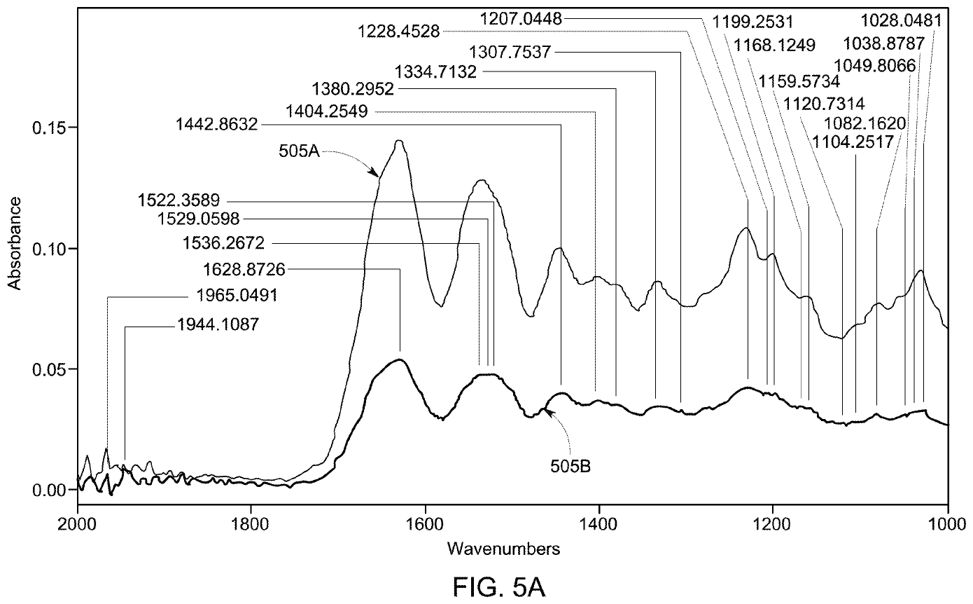

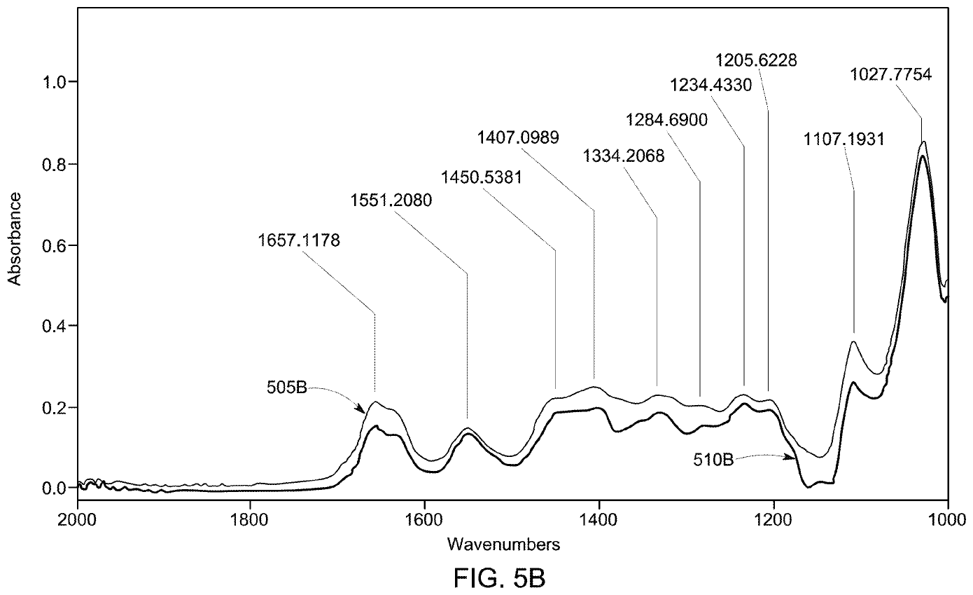

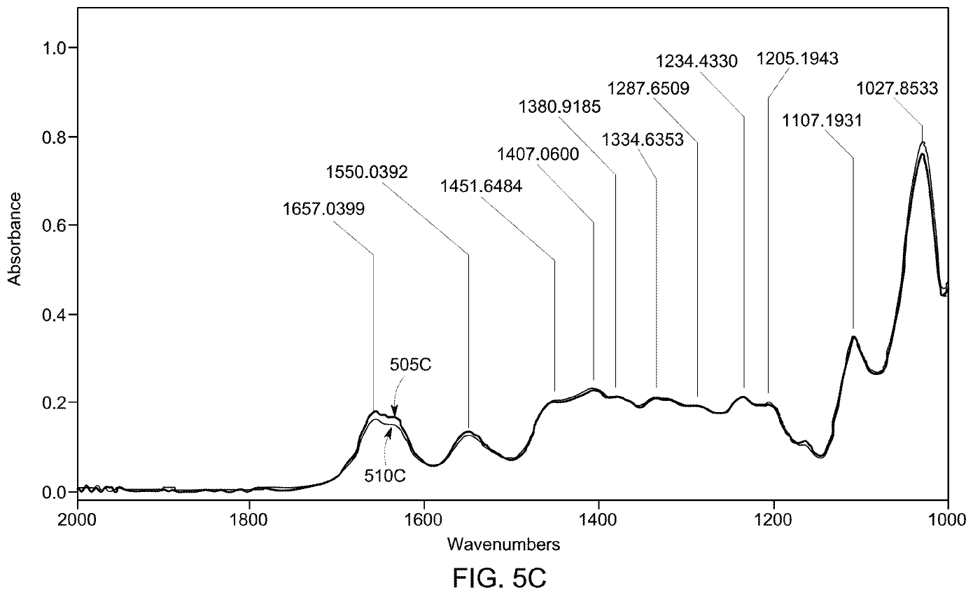

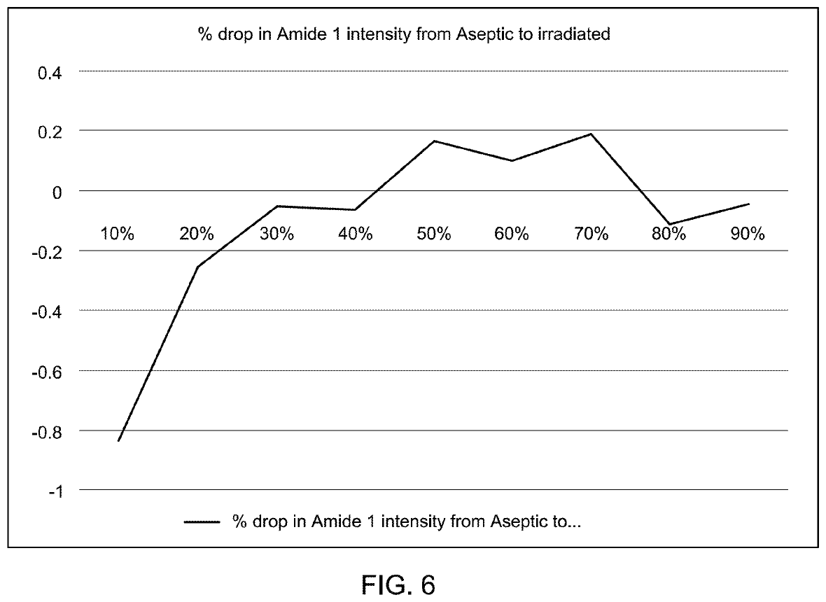

FIGS. 5A, 5B and 5C are graphs showing peak positions of solutions with 0% glycerol protectant, 20% glycerol protectant, and 50% glycerol protectant, respectively. FIG. 6 is a graph showing protection of amide intensity with active protection technology when irradiated with gamma irradiation.

Among the groups, 0% (no protection) was observed to have a different peak position compared to that expected for amide I, as illustrated in FIG. 5A. It is understood to have a different conformation due to the collapse of the collagen structure as it is dehydrated and interpeptide bonds form. With solutions of 10% to 20% concentration of glycerol in alcohol, it was observed that the irradiation significantly damaged the secondary structure of collagen as shown by a drop in the amide 1 peak intensity in FIG. 5B. As the concentration of the protectant (glycerol) in solvent (alcohol) is increased beyond 20%, the damage to the secondary structure decreased sharply. The secondary structure damage was completely prevented when the glycerol concentration is further increased beyond 40% up to 70%, as shown in FIG. 5C. In the descriptions provided herein, reversed means that a damaged structure was treated with the protectant and the damage went away and protected means that treatment with the protectant prevented damage from just the one treatment.

In this example, beyond a 70% glycerol concentration, the handling of the samples deteriorates. Additionally, beyond 80%, the protection also becomes less effective. This may be seen from the test results summarized in TABLE 2 below, which provides peak intensities at different protectant concentrations. This may occur due to higher surface tension of the solution at a higher concentration that fails to penetrate deep enough within the matrix to replace the collagen water. Therefore, a clear window of processing emerged with the protectant technology that establishes an optimum concentration of glycerol in alcohol to be around 30%-70%, with the best results being between 45% to 60%. This range may provide the best handling as well as best protection from terminal irradiation.

TABLE 2

| Glycerol | | | | % drop in Amide 1 |
|---|---|---|---|---|
| Conc. In | Aseptic | Irradiated | Peak | intensity from |
| alcohol | intensity | intensity | position | Aseptic to irradiated |
| *0% | 5.4717 | 2.1882 | 1628.8726 | −60% |
| 10% | 18.0672 | 3.0166 | 1651.4103 | −83% |
| 20% | 9.2118 | 6.8809 | 1657.1178 | −25% |
| 30% | 10.0634 | 9.4962 | 1656.4165 | −6% |
| 40% | 10.189 | 9.5195 | 1656.884 | −7% |
| 50% | 7.113 | 8.2743 | 1657.0309 | 16% |
| 60% | 7.1293 | 7.8247 | 1657.1178 | 10% |
| 70% | 9.6055 | 11.4137 | 1657.3516 | 19% |
| 80% | 10.3951 | 9.1999 | 1656.2217 | −11% |
| 90% | 8.6347 | 8.2332 | 1656.884 | −5% |
| *100% | 6.9719 | 8.7943 | 1657.7606 | 26% |

For other collagen sources and types, the optimum concentration ratio of glycerol to alcohol may be different as influenced by the microstructure of the collagen. This optimum concentration may also depend on the particular solvent/solvent mix and protectant as well as the matrix that is being treated. However, the same principles described herein may be used to determine the optimum ratio for any type of collagen. Particularly, the type of collagen (e.g., bovine or human), gender of the donor, age and size may be used to determine the optimal ratio for a particular collagen. It may also be possible that employing processing aids, such as pulsed vacuum or ultrasonic agitation, may vary the concentration range at which glycerol in alcohol maintains its effectiveness.

In another experiment, a mixed solvent was used. In this example, an alcohol mixture of isopropyl alcohol and methanol was used as a solvent and glycerol was used as a protectant. Bovine fibers and particulates were both used to prepare the samples.

A first sample of DBM putty was prepared by milling fresh bovine tissue into fibers and demineralizing them. The tissue was treated with an active protectant solution, which was prepared by mixing a 40% (v/v) concentration of the protectant with the solvent. The tissue was vacuum dried after protectant treatment and immediately double bagged for gamma irradiation.

A second sample of 100% DBM fibers was prepared by milling fresh bovine tissue into fibers and demineralizing them. The fibers were not treated with the active protectant solution, but the tissue was directly vacuum dried afterwards and immediately double bagged for gamma irradiation.

A third sample of DBM gel was prepared by granulating fresh bovine tissue into particles and demineralizing them. The tissue was treated with treated with an active protectant solution, which was prepared by mixing a 40% (v/v) concentration of the protectant with the solvent. The tissue was vacuum dried after protectant treatment and immediately double bagged for gamma irradiation.

The samples were split into two parts for irradiation. One part of the sample from each group was sterilized by gamma irradiation at a dose of 25-35 kGy on dry ice. The other part was not irradiated. It constituted the aseptic samples. Both the aseptically prepared samples as well as the terminally irradiated samples were held at room temperature for 6 months before commencing evaluation of the properties. Descriptions of the samples and groups are provided in TABLE 3 below.

Measurement of increased ALP expression enzymatically may be considered to be a reliable early marker of the osteoblastic phenotype as undifferentiated mesenchymal stem cells show weak alkaline phosphatase activity. The osteogenic differentiation of mesenchymal stem cells in vitro has been divided into three stages. During the initial stage (days 1-4), proliferation of cells is dominant, which is followed by early cell differentiation into osteoblasts, which is characterized by the transcription and protein expression of alkaline phosphatase. After this initial peak of ALP, its level starts to decline.

In the experiment, three different forms of products with bovine tissue, namely, putty, fibers, and gel, each in aseptic and irradiated form, were prepared with glycerol as the protectant and anhydrous alcohol as the solvent in the putty and gel samples (A1, A3 and B1, B3). Dry fiber without any active protection was prepared as a control (A2, B2). A brief description of the preparations is described above and is summarized in TABLE 3 below. Equal weights of each sample (67 mg) were plated in 48 well plates. Pluripotent Human Bone Marrow Derived Mesenchymal Stem Cells; Human (ATCC® PCS 500012™) were seeded on the scaffolds and incubated in the presence of the DBM samples for 5 and 7 days. The media was sampled at 5 and 7 days. A standard colorimetric alkaline phosphatase assay (alkaline phosphatase kit from EMD Millipore, Billerica, Mass.) was run on the preserved media along with controls.

TABLE 3

| Gr. | Sample | Sample | |
|---|---|---|---|
| # | codes | description | Type |
| 1 | A1 | Aseptic DBM putty with | Aseptic fiber with |
| | | active protection | glycerol as protectant |
| 2 | A2 | Aseptic dry DBM without | Dry aseptic fiber |
| | | active protection | |
| 4 | A3 | Aseptic injectable DBM | Aseptic particles with |
| | | Gel with active protection | glycerol as protectant |
| 5 | B1 | Terminally irradiated | Terminally irradiated |
| | | DBM putty with active | fiber with glycerol as |
| | | protection | protectant, Sterilized |
| | | | @25-35 kGy with |
| | | | dry ice |
| 6 | B2 | Terminally irradiated | Terminally irradiated |
| | | dry DBM without active | fiber, 25-35 kGy with |
| | | protection | dry ice |
| 8 | B3 | Terminally irradiated | Terminally irradiated |
| | | injectable DBM Gel particles | particles, with glycerol |
| | | with active protection | as protectant, Sterilized |
| | | | @25-35 kGy with dry |
| | | | ice |

Colorimetric absorbance values measured at 405 nm were expressed as mean±standard deviation. The differences between the groups were analyzed by ANOVA followed by a post hoc Tukey's test. Differences were considered significant at p≤0.05. Data were analyzed using Minitab 17.2 (Minitab Inc., State College Pa., USA).

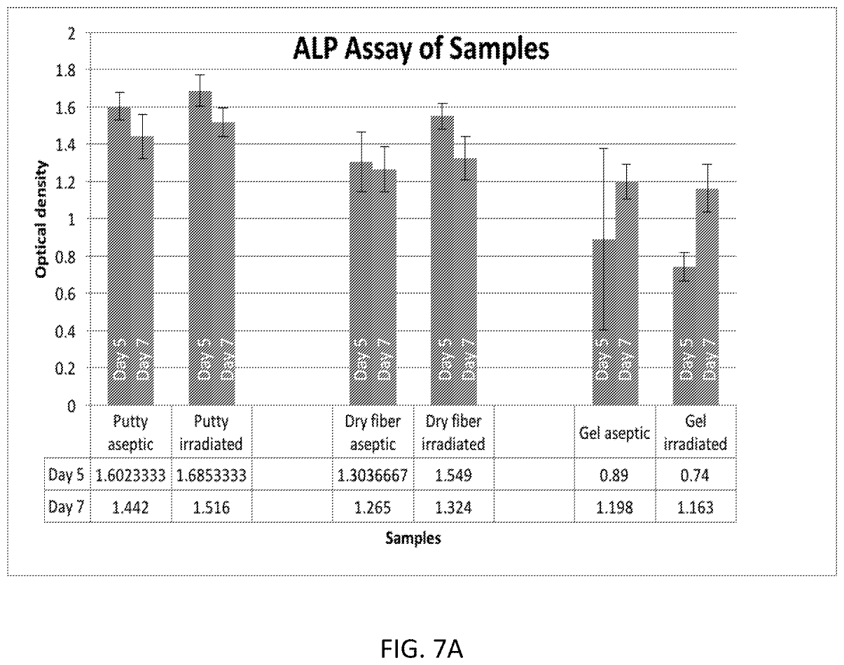

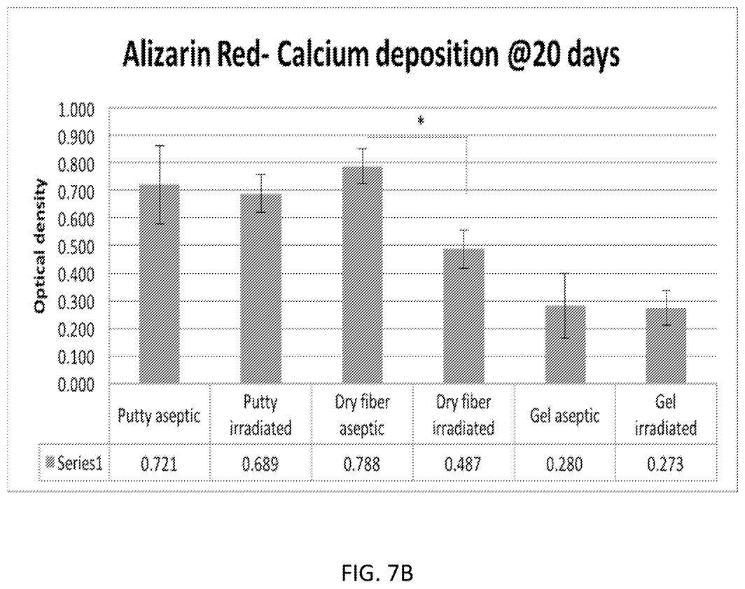

FIG. 7A is a graph showing the ALP assay of aseptic and terminally irradiated samples at 5 and 7 days. As shown in FIG. 7A, fiber based products showed a decline of ALP activity from the 5th to 7th day, signifying an onset of differentiation of mesenchymal stem cells at this point. However, particulate based products still showed a continuing increase in ALP activity during the same time frame, signifying a comparatively later differentiation on the particulate based scaffolds. Particulate based scaffolds are comparatively more labile, which may disturb the proliferation and differentiation of the same. Nevertheless, in the long term, the mineralization study demonstrates differentiation on both types of scaffolds.

A comparison between the aseptic and the irradiated products within the same groups and same time points in FIG. 7A showed that none of the groups were significantly different from each other. This may signify that there is no difference in performance between the aseptic and irradiated samples at the 5 and 7 day time points. It was also observed that both the dried fiber samples (irradiated and aseptic) as described in Table 2 above, had statistically the same ALP scores compared to their corresponding (glycerol treated) putty samples.

The aseptic treatments with the tissue showed that the dry aseptic fiber had equivalent ALP activity compared to aseptically processed tissue with active protection. It may be noted that the all the samples had the same weight at plating. Therefore, the putty formulations with active protection had a lower amount of DBM fibers, with the protectant taking up about half of the weight compared to the DBM dry fibers. Therefore, the putty formulations with active protection had the same activity and so outperformed the tissue without active protection because the protected tissue had about half as much tissue content as the dry fiber samples. This demonstrates that the active protection treatment preserves activity better even in case of aseptic tissue processing as well as during irradiation of the tissue.

ALP as a marker is essential for bone development but it is not unique to Osteoblasts. There are multiple cell types/lines that secrete ALP. However, calcium deposition is unique to osteoblasts. Therefore, a second confirmation of the differentiation of stem cells to osteoblasts may be needed. Direct staining of extracellular calcium deposits (mineralization) or colorimetric assay to determine calcium quantitatively confirms differentiation of mesenchymal stem cells into osteoblasts. In contrast to undifferentiated mesenchymal stem cells, differentiated osteoblasts accumulate vast extracellular calcium deposits (mineralization). This process is accompanied by the formation of bone nodules. Osteoblast-mediated mineralization is therefore indicative of the formation of bone mass and can be specifically detected using the bright orange-red dye, Alizarin Red S.

In this experiment, comparative amounts of mineralization in the scaffolds by the differentiated cells in example 4 were assessed by a quantitative Alizarin Red S method at 20 days. Cells were fixed and stained with 2% Alizarin Red S (Sigma Aldrich) for approximately 10 minutes. Quantification of the stain was performed by extracting the stain. The extract was quantified calorimetrically by measuring the absorbance at 570 nm. Absorbance values were expressed as mean±standard deviation. The differences between the groups were analyzed by ANOVA followed by a post hoc Tukey's test. Differences were considered significant at p≤0.05. Data were analyzed using Minitab 17.2 (Minitab Inc., State College Pa., USA). FIG. 7B is a graph showing the mineralization assay of aseptic and terminally irradiated samples.

Mineralization is the definitive test for the bone forming cells called osteoblasts. The pluripotent mesenchymal stem cells seeded on the scaffold are differentiated specifically to osteoblasts due to the effect of osteoinductive factors present in the scaffolds post processing. Experimental results for the different groups for the mineralization assay show the effect of active protection on the osteoinductive factors of the scaffolds during processing and irradiation. These essential factors for irradiated putty and irradiated gel are better protected compared to the dry fibers during irradiation without the active protection at a 20 day time point.

It was observed that there was no statistical difference between the fiber based aseptic putty and the actively protected irradiated putty regarding mineralization. The particulate based Gel product also acted in a similar fashion with no statistical difference in mineralization between the aseptic sample and the actively protected irradiated sample. However, the dry fiber irradiated without any active protection showed significantly lower mineralization and calcium deposition after 20 days compared to the aseptically prepared dry fiber. This signifies that drying alone may not be sufficient to protect the tissue from damage which is manifested in poor mineralization of the scaffold during healing.

It may be noted that all the grafts were made from the same batch of tissue. The putty formulation (both aseptic and irradiated) contains only ˜50% tissue weight while the rest is glycerol. However, this formulation performed equivalent to the aseptic dry fiber formulation, which contains 100% tissue. Therefore, it may be concluded that the actively protected putty formulation conserves the scaffold activity better and is capable of performing better, even at a lower dose.

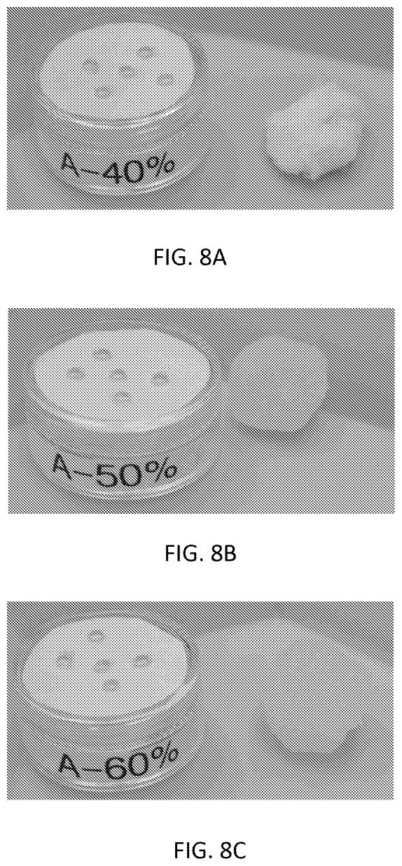

As mentioned above, DBM may be actively protected by exposing the DBM to an amount of a mixture of an active protectant and a solvent. The amount of the mixture to which the DBM is exposed during processing, as well the drying process, and potentially other factors, may determine a concentration of active protectant (e.g., glycerol) to bone that is included in the completed product. FIGS. 8A, 8B and 8C are pictures showing a packaged and terminally irradiated DBM product that includes a 40%, 50% and 60% glycerol to bone concentration, respectively. As can be seen from the pictures, the 40% by concentration sample in FIG. 8A appears drier than the 50% and 60% by concentration samples in FIGS. 8B and 8C. If the concentration of protectant in the final product is too low, the DBM may be too fragile for practical use, and if the concentration of protectant in the final product is too high, the DBM may be too viscous for practical use. The above-mentioned factors may, therefore, be chosen to provide an optimal concentration of protectant in the final product. In embodiments, a final DBM product having between 40% and 60% or optionally 50% concentration of protectant may be chosen.

What is claimed is:

1. A method of protecting a Demineralized Bone Material (DBM) against damage caused by terminal irradiation, the method comprising:
providing the DBM, the DBM comprising at least a collagen matrix and natural, non-collagenous protein;
removing and replacing water in the collagen matrix of the DBM by soaking the DBM in a mixture of glycerol and a solvent; and
drying the DBM after the soaking to form a protected DBM.

2. The method of claim 1, wherein the mixture contains between 30% and 70% glycerol by volume.

3. The method of claim 2, wherein the mixture contains between 45% and 60% glycerol by volume.

4. The method of claim 1, wherein the mixture contains 50% glycerol by volume.

5. The method of claim 1, wherein the solvent contains at least one of isopropyl alcohol, acetone and methanol.

6. The method of claim 1, further comprising:
packaging the protected DBM in a sterile pouch to form a packaged DBM.

7. The method of claim 6, further comprising:
terminally sterilizing the packaged DBM by exposing the packaged DBM to one of gamma and electron-beam radiation.

8. The method of claim 1, wherein the DBM is in a form of a putty, a gel or fibers.

9. The method of claim 1, wherein the protected DBM contains greater than 50% glycerol by weight.

10. The method of claim 1, wherein the soaking the DBM in the mixture of glycerol and the solvent is performed under a pressure of 40-100 kPa.

11. A system for protecting a Demineralized Bone Material (DBM) against damage caused by terminal irradiation, the system comprising:
a plurality of valves that control a flow of a mixture of glycerol and a solvent;
a reaction chamber that receives the DBM and the flow of the mixture;
a drier; and
a controller that is communicatively coupled to the plurality of valves and the drier, wherein the controller:
soaks the DBM with the mixture in the reaction chamber by controlling the plurality of valves, the DBM comprising at least a collagen matrix and natural, non-collagenous protein, whereby water is removed and replaced in the collagen matrix of the DBM as a result of the soaking,
removes the mixture from the reaction chamber by controlling the plurality of valves, and
dries the DBM to form a protected DBM by controlling the drier.

12. The system of claim 11, wherein the mixture contains between 30% and 70% glycerol by volume.

13. The system of claim 11, wherein the mixture contains between 45% and 60% glycerol by volume.

14. The system of claim 11, wherein the mixture contains 50% glycerol by volume.

15. The system of claim 11, wherein the solvent contains at least one of isopropyl alcohol, acetone and methanol.

16. The system of claim 11, wherein the DBM is in a form of a putty, a gel or fibers.

17. The system of claim 11, wherein the protected DBM contains greater than 50% glycerol by weight.

18. A terminally sterilized DBM formed by the method of claim 1.

19. The terminally sterilized DBM of claim 18, wherein the sterilized DBM is a form of a putt, a gel or fibers.

20. A terminally sterilized DBM formed by the method of claim 7.
